(19) United States
(12) Patent Application Publication
(10) Pub. No.: US 2025/0369954 A1
(43) Pub. Date: Dec. 4, 2025

(54) PLATFORM AND METHOD TO CONTINUOUSLY MONITOR FUNCTIONALITY OF CARDIOMYOCYTES OR OTHER CELLS ON BOTH SIDES OF A POROUS MEMBRANE

(71) Applicant: Government of the United States of America, as represented by the Secretary of Commerce, Gaithersburg, MD (US)

(72) Inventor: Darwin René Reyes-Hernández, Clarksburg, MD (US)

(21) Appl. No.: 19/220,971

(22) Filed: May 28, 2025

Related U.S. Application Data
(60) Provisional application No. 63/652,428, filed on May 28, 2024.

(51) Int. Cl. G01N 33/50; C12M 1/12; C12M 1/34; C12M 1/42; C12M 3/06

(52) U.S. Cl. CPC G01N 33/5023; C12M 23/16; C12M 25/02; C12M 35/02; C12M 41/46; G01N 33/5061

(57) ABSTRACT

A platform with unique integrated electronic detection format to enable measurement of cell responses to chemical and physical stimulation on both sides of a porous membrane is disclosed. This sensing platform supports measurements including impedance measurements to determine physical displacement of cells and bioelectrical activity detection of cell-cell chemical induced responses in cardiomyocytes. Advantageously, cardiac cells can be positioned on opposite sides of a porous membrane and can be independently stimulated and monitored. Additionally, cardiac cells or other electrically active cells can be positioned and monitored on one side of the porous membrane, while other cells such as endothelial cells, epithelial cells, or liver cells may be positioned and monitored on the other side of the porous membrane.

### CROSS REFERENCE TO RELATED APPLICATIONS

This application claims the benefit of U.S. Provisional Application Ser. No. 63/652,428 (filed May 28, 2024) which is herein incorporated by reference in its entirety.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### COPYRIGHT NOTICE

This patent disclosure may contain material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Organ-on-a-chip (OOC) type devices simulate the activities, mechanics, and responses to chemical and physical stimulation of organs or cells and can be used in biomedical engineering research. Efforts towards observing interactions between cells of the same type or different types remains a subject of ongoing research. These OOC devices include heart-on-a-chip systems intended to assist researchers investigate cardiovascular diseases, which are one of the leading causes of death worldwide. However, the integration of electrical and electronic sensors of cell monitoring within these systems remains a formidable challenge, especially in multi-channel and multi-layer architectures.

One type of OOC device for observing interactions between cells involves a device with a porous membrane separating the cell types and means for electronic detection of cell behavior on one side of the membrane. This setup allows for the detection of cell behavior on one side of the membrane. However, existing methods do not provide for real-time electronic detection of cell behavior on both sides of the membrane. Similarly, existing methods and platforms do not enable arrays of electrodes to both electronically manipulate and measure both sides of a membrane.

The present invention generally relates to an organ-on-a-chip platform enabling electronic manipulation and measurement of cells on both sides of a membrane.

### BRIEF DESCRIPTION

The present invention contemplates a platform to continuously measure changes in beating rate and beating amplitude (seen as changes in impedance and voltage) of cardiac cells under myocardial infarction to determine changes in cell function. Cardiac cells can be positioned on opposite sides of a porous membrane and cells can be independently stimulated on one side to experience cardiac infarction (i.e., hypoxia) while the opposite side is under normal conditions. Communication between infarcted and healthy cells on opposite sides of the membrane can be tracked using electronic measurements. This enables real-time electronic detection of changes in cell behavior on both sides of the membrane in a way not possible with existing organ-on-a-chip (OOC) devices.

Additionally, the invention includes the ability to simultaneously measure TransEpithelial/Endothelial Electrical Resistance (or TEER) on cells forming a cell layer that is completely sealed (i.e., cell-cell adhering through cell-cell junctions), while cardiac cells are monitored continuously on the opposite side (or neural cells or any other cell whose behavior can be measured electronically, though the cells are not necessarily electroactive, via impedance or other type of electronic measurement). Therefore, this system allows for two simultaneous different electronic measurements when two different cell types, for example (but not limited to), endothelial cells and cardiac cells or liver and cardiac cells, are placed within this device. On one side TEER measurements are used to monitor the interaction of endothelial or epithelial cells with drugs or toxicants that produce a disruption of the cell-cell junctions or a change in the monolayer of the endothelial/epithelial cells that produces a change in the TEER measurement. At the same time, the beating of cardiac cells is also monitored continuously to measure changes due to the disruption of cell-cell junctions and/or the release of chemicals from the cells exposed to drugs or toxicants (e.g., chemical compounds).

To enable the use of such a platform, a process has been developed to fabricate electrodes directly on porous and biocompatible membranes, where cells such as cardiomyocytes can then be cultured directly on the membrane, allowing for continuous, real-time monitoring of cell behavior.

Disclosed is a platform enabling electronic manipulation and measurement of cells on both sides of a membrane, the platform comprising: a porous membrane, a first set of microfabricated electrodes disposed on a first side of the membrane, a second of microfabricated electrodes disposed on a second side of the membrane, a first substrate with microfluidic channels creating a bottom flow channel disposed on the first side of the membrane, and a second substrate with microfluidic channels creating a top flow channel disposed on the second side of the membrane; wherein the porous membrane is biocompatible.

Further disclosed is a method for fabricating a platform to continuously monitor functionality of cells on both sides of a porous membrane, the method comprising: forming pores in a membrane material to create a porous membrane; performing a first electrode fabrication process to create a first set of electrodes on a first processed side of the membrane comprising: attaching the membrane material to a supporting structure, coating the side of the membrane material opposite the supporting structure with a lift-off photoresist material, transferring electrode patterns to the lift-off photoresist material by photolithography exposure, removing residual lift-off photoresist material from the membrane material, metallizing the membrane material with a conductive metal by a metal deposition process, rinsing to remove the lift-off photoresist material and the excess conductive metal, and removing the membrane material from the supporting structure; performing a second electrode fabrication process to create a second set of electrodes on a second processed side of the membrane by attaching the supporting structure to the first processed side and repeating the remaining steps of the first electrode fabrication process; fabricating a first master mold with the negative of a first microfluidic channel; using the first master mold to transfer the pattern of the first microfluidic channel into a first substrate; bonding the first substrate to the first processed side; fabricating a second master mold with the negative of a second microfluidic channel; using the second master mold to transfer the patter of the second microfluidic channel into a second substrate; and bonding the second substrate to the second processed side; wherein the first set of electrodes allow connection with the porous membrane area on the first processed side; and wherein the second set of electrodes allow connection with the porous membrane on the second processed side.

Also disclosed is a method for electronic manipulation and measurement of cells on both sides of a membrane, the method comprising: providing a microfluidic platform with integrated electronic sensors, itself comprising: a porous membrane, a first set of microfabricated electrodes disposed on a first side of the membrane, a second of microfabricated electrodes disposed on a second side of the membrane, a first substrate with microfluidic channels creating a bottom flow channel disposed on the first side of the membrane, and a second substrate with microfluidic channels creating a top flow channel disposed on the second side of the membrane; and guiding and capturing a first cell type on the first set of electrodes on the first side of the membrane.

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows, according to some embodiments, a microfluidic device with integrated electronic sensors and microfluidic channels on both sides of a porous membrane.

FIG. 2 shows, according to some embodiments, a cross-section of a microfluidic device showing the flow in microfluidic channels on both sides of a porous membrane with integrated electronic sensors.

FIG. 3 shows, according to some embodiments, measured fluorescence intensity from a voltage-sensitive dye representing the visually measured contraction rate of cardiomyocytes derived from human induced pluripotent stem cells.

FIG. 4 shows, according to some embodiments, a Bode plot of the electrodes on one side of a microfluidic device used for impedimetric characterization of cells captured on the membrane.

FIG. 5 shows, according to some embodiments, the change in impedance of cardiomyocytes representing beating before (top) and after (bottom) treatment with verapamil, demonstrating cessation of cardiomyocyte beating.

FIG. 6 shows, according to some embodiments, a representation of an experimental setup using cardiomyocytes on one side of the membrane and human umbilical vein endothelial cells on the other side, with the potential diffusion of cardioactive drugs across the endothelium represented by a solid arrow and flow direction in the channels represented by hollow arrows.

### DETAILED DESCRIPTION

A detailed description of one or more embodiments is presented herein by way of exemplification and not limitation.

Advantageously, a microfluidic device has been developed which can serve as a platform for continuously monitoring the functionality of cells on both sides of a porous membrane. This uniquely allows for the electronic manipulation and measurement of the properties of cells on both sides of the membrane. These cells can be cultured directly on the membrane by using the integrated electrodes of the device to enable dielectrophoretic cell capture or by filling the channel with cells and allowing them to settle down and attached on their own on the membrane surface. Having separate microfluidic channels and electrodes on both sides of the membrane enables the same or different cell types to be co-cultured on opposite sides of the membrane, allowing for observations related to the interaction between the cell types across the porous membrane.

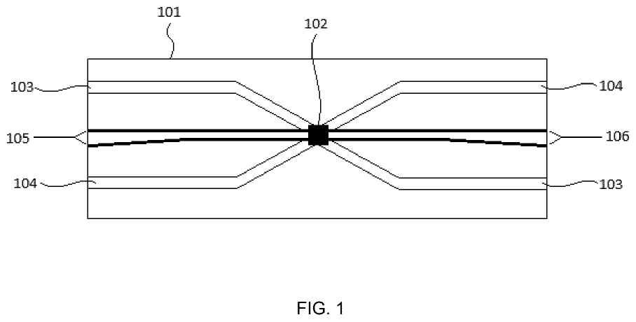

As shown in FIG. 1, in one embodiment, a microfluidic device 101 containing a porous membrane exchange area 102 is shown with microfluidic channels and interdigitated electrodes on both sides of the membrane. Microfluidic channel 103 and interdigitated electrode 105 is disposed on one side of the membrane, with the electrode 105 reaching the porous membrane exchange area 102 on one side and microfluidic channel 103 crossing the same side of the porous membrane area. Similarly microfluidic channel 104 and interdigitated electrode 106 reach and cross porous membrane exchange area 102 on the opposite side of the membrane.

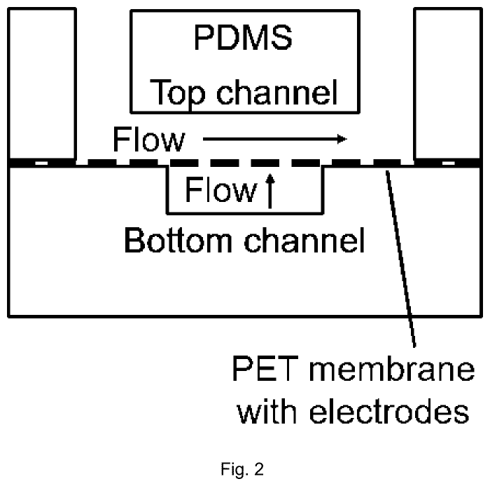

As shown in FIG. 2, in one embodiment, the microfluidic device comprises a polyethylene terephthalate (PET) membrane with integrated electrodes, a top section of polydimethylsiloxane (PDMS) with an integrated microfluidic top channel crossing the top of the membrane, and a bottom PDMS section with an integrated microfluidic bottom channel crossing the bottom of the membrane. While the figure describes a PET membrane, other suitable materials may be used, including polycarbonate or any other material on which pores can be fabricated while maintaining mechanical stability. While the figure describes the material with integrated channels as being PDMS, other suitable materials may be used including, but not limited to, thermoplastics (e.g. polystyrene, polymethyl methacrylate, PMMA, CoC and others) and glass.

In one embodiment the availability of a microfluidic channel and integrated electrodes on one side of the porous membrane allows for the dielectrophoretic cell capture of cells directly on the portion of the membrane to be measured by the integrated electrode. The availability of similar channel and electrodes on the other side of the porous membrane allows for the capture of the same or different cell type on that side of the membrane.

With the use of optically transparent materials such as PET for the membrane and PDMS for the sections including the microfluidic channels, simultaneous optical imaging methods are possible, such as the use of optical microscopy to monitor immunohistochemical staining, voltage sensitive dyes, or other optical markers in real time.

The systems and processes herein are illustrated further by the following Examples, which are non-limiting.

### Example 1—Culture of hiPSC-CM on Microfluidic Device

In one embodiment, human induced pluripotent stem cell-derived cardiomyocytes (hiPSC-CM) were cultured directly on the PET membrane of the microfluidic device. In this example, immunohistochemical staining was used to confirm both the expression of pluripotency markers in the captured hiPSC, and pluripotency markers OCT4 and SSEA4 were demonstrated. Later, the successful differentiation of the hiPSC into cardiomyocytes was also demonstrated with the expression of cardiac markers NKX2-5 and TNNT2.

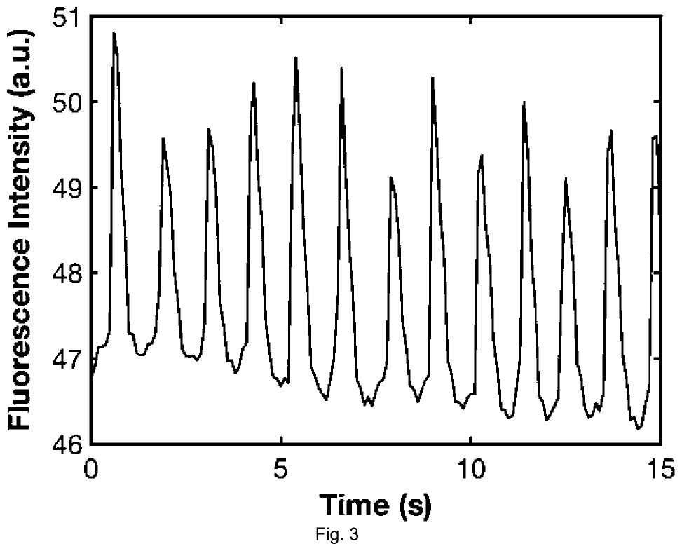

Approximately 10-15 days after commencing differentiation, spontaneous contraction of individual cardiac syncytia was seen. Synchronized activity measured with a voltage-sensitive fluorescent probe was also used to determine contraction frequency, which in this example was approximately 50 beats per minute as shown in FIG. 3.

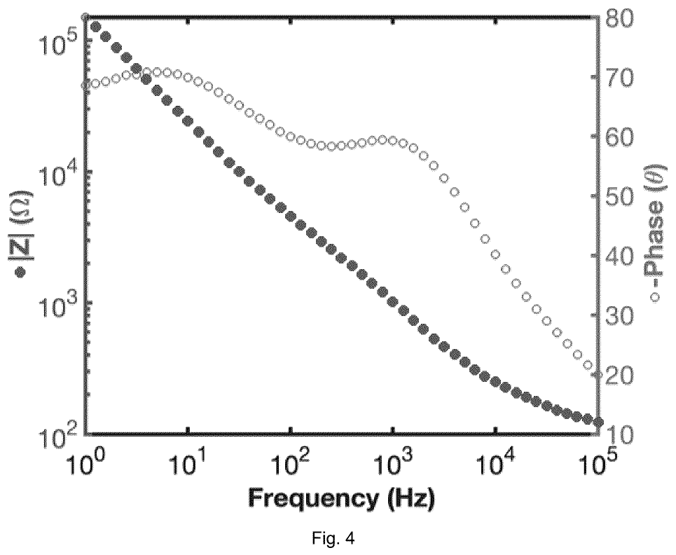

Cells in this example were trapped in the region of interest within the device using dielectrophoretic cell capture, directing cells from the microfluidic channel to the electrode. In this example, the entire electrode was covered with cells in increasing density from around 1 to 3 minutes. The following parameters for dielectrophoresis were used in this example: 4-6 Vpp, 12 MHz, 10-50 10-50 μl min-1. A Bode plot of the electrodes after the cell capture shows impedimetric characterization of the cells, as show in FIG. 4.

After the cells form a contracting syncytium, the electrodes can also be used for electrical pacing of the hiPSC-CMs. This pacing is a potential technique for the on-chip maturation of the cells.

### Example 2—Bioelectrical Activity Detection of Cell-Cell Chemical Induced Responses

In myocardial infarction, cardiac cells die as oxygen levels decrease due to arterial blockage. This event triggers a change in the beating rate of cardiac cells that limits the function of the heart to a point that it could be fatal. Efforts to regenerate heart function using cell-based therapies suggests that cell signaling molecules, rather than the transplantation of entire stem cells onto myocardial infarcted cells, may be responsible for the improvement in observed heart function. Because this mechanism of action is not well understood, a platform to measure relevant changes is needed.

In one embodiment, the microfluidic device can be configured to measure changes in beating rate and amplitude (seen as changes in impedance and voltage) of cardiac cells under myocardial infarction to determine if improvements in cell function are due to cell signaling molecules. Continuous monitoring of cell behavior will also allow monitoring of the changing aspects of this interaction. Cardiac cells are positioned on opposite sides of the porous membrane and cells on one side are stimulated to experience cardiac infarction (i.e., hypoxia). Communication between infarcted and healthy cells on opposite sides of the membrane occurs through the release of cell signaling molecules, i.e., exosomal secretions. The exosomal secretions are fluorescently monitored by staining components of the exosomes or the exosome membrane itself.

Other measurement methods using this organ on a chip-type device do not provide real-time electronic detection of changes in cell behavior on both sides of the membrane; the microfluidic platform addresses this deficiency.

### Example 3—Simultaneous Measurement of TransEpithelial/Endothelial Electrical Resistance (TEER)

In one embodiment, a hiPSC-CM culture is captured on one side of the porous membrane of the microfluidic device as in Example 1. Other cells related to heart vasculature may then be captured on the opposite side of the porous membrane to mimic certain aspects of heart vasculature for investigation. In this example, endothelial cells are captured on the opposite side of the porous membrane. In this configuration, simultaneous measurements are possible from cardiomyocytes on one side of the membrane (such as beating frequency and amplitude), while measurements such as TransEpithelial/Endothelial Electrical Resistance (TEER) can be taken from the endothelial cells.

### Example 4—Treatment of Cardiomyocytes with Cardioactive Drug

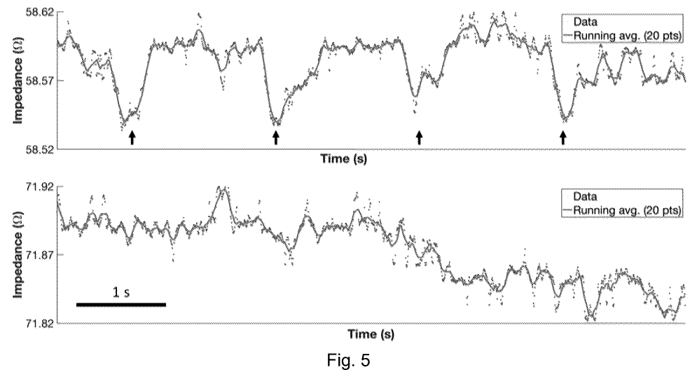

In one embodiment, hiPSC-CM cells are captured on one side of the porous membrane as in Example 1. The cardiomyocytes are treated with 1 μM verapamil, a Ca²⁺ channel blocker used for treating arrhythmia. As show in FIG. 5, impedance measurements of the iPSC-CM cells before treatment (top figure) showed values consistent with the cells beating, while measurement of the cells after 10 minutes of treatment demonstrated a cessation of beating (bottom figure).

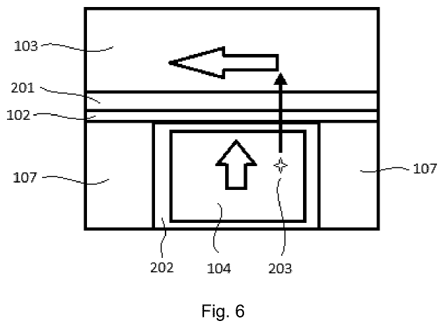

In an extension of this example, the effect drug diffusion across endothelium can also be investigated. As shown in FIG. 6, hiPSC-CM cells 201 can be captured on the porous membrane 102 in the first channel 103, while human umbilical vein endothelial cells (HUVECs) 202 are captured in the second channel 104. For clarity, flow direction of the channels are represented by hollow arrows and are approximately parallel to the porous membrane, while the substrate of the second channel is also shown 107. A cardioactive drug 203 may be introduced in the second channel 202, cross the endothelial cell layer 202 and the porous membrane 102 and may interact with the cardiomyocyte layer 201. This setup will allow for measurements of the hiPSC-CM cells similar to those shown in FIG. 5, as well as potential changes to the endothelium layer.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix(s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier “about” used in connection with a quantity is inclusive of the stated value and has the meaning dictated by the context (e.g., it includes the degree of error associated with measurement of the particular quantity). The conjunction “or” is used to link objects of a list or alternatives and is not disjunctive; rather the elements can be used separately or can be combined together under appropriate circumstances.

What is claimed is:

1. A platform enabling electronic manipulation and measurement of cells on both sides of a membrane, the platform comprising:
a porous membrane,
a first set of microfabricated electrodes disposed on a first side of the porous membrane,
a second set of microfabricated electrodes disposed on a second side of the porous membrane,
a first substrate with microfluidic channels creating a bottom flow channel disposed on the first side of the porous membrane, and
a second substrate with microfluidic channels creating a top flow channel disposed on the second side of the porous membrane;
wherein the bottom flow channel and the top flow channel cross on opposite sides of the porous membrane, creating a membrane exchange area.

2. The platform of claim 1, wherein the porous membrane is a biocompatible material.

3. The platform of claim 2, wherein the porous membrane is polyethylene terephthalate.

4. The platform of claim 1, wherein the first set and the second set of microfabricated electrodes are interdigitated on opposite sides of the membrane exchange area.

5. A method for fabricating a platform enabling electronic manipulation and measurement of cells on both sides of a membrane, the method comprising:
forming pores in a membrane material to create a porous membrane;
performing a first electrode fabrication process to create a first set of electrodes on a first processed side of the membrane comprising:
attaching the porous membrane to a solid supporting structure,
coating the side of porous membrane opposite the supporting structure with a lift-off photoresist material,
transferring electrode patterns to the lift-off photoresist material by photolithography exposure,
removing residual lift-off photoresist material from the porous membrane,
metallizing the porous membrane with a conductive metal by a metal deposition process,
rinsing to remove the lift-off photoresist material and the excess conductive metal,
and removing the porous membrane from the supporting structure;
performing a second electrode fabrication process to create a second set of electrodes on a second processed side of the porous membrane by attaching the supporting structure to the first processed side and repeating the remaining steps of the first electrode fabrication process;
fabricating a first master mold with the negative of a first microfluidic channel;
using the first master mold to transfer the pattern of the first microfluidic channel into a first substrate;
bonding the first substrate to the first processed side with the first microfluidic channel oriented towards the first processed side;
fabricating a second master mold with the negative of a second microfluidic channel;
using the second master mold to transfer the patter of the second microfluidic channel into a second substrate; and
bonding the second substrate to the second processed side with the second microfluidic channel oriented towards the second processed side;
wherein the first microfluidic channel and the second microfluidic channel cross on opposite sides of the porous membrane, creating a membrane exchange area;
wherein the first set of electrodes allow connection in the membrane exchange area on the first processed side; and
wherein the second set of electrodes allow connection in the membrane exchange area on the second processed side.

6. The method of claim 5, wherein the membrane material is selected from a list comprising polyethylene terephthalate and polycarbonate.

7. The method of claim 5, wherein the first substrate and second substrate are selected from a list comprising: polydimethylsiloxane, polystyrene, polymethyl methacrylate, or a cyclic olefin copolymer.

8. The method of claim 5, wherein pores are formed in the entire membrane material.

9. The method of claim 5, wherein pores are formed only in the membrane exchange area.

10. The method of claim 5, wherein the first and second set of electrodes are interdigitated on opposite sides of the membrane exchange area.

11. The method of claim 5, wherein the first and second master molds are selected from a list comprising metal, silicon, glass, or thermoplastic.

12. A method for electronic manipulation and measurement of cells on both sides of a membrane, the method comprising:
providing a platform with integrated electrodes, itself comprising:
a porous membrane,
a first set of microfabricated electrodes disposed on a first side of the porous membrane,
a second set of microfabricated electrodes disposed on a second side of the porous membrane,
a first substrate with microfluidic channels creating a bottom flow channel disposed on the first side of the porous membrane, and
a second substrate with microfluidic channels creating a top flow channel disposed on the second side of the porous membrane,
wherein the bottom flow channel and the top flow channel cross on opposite sides of the porous membrane, creating a membrane exchange area; and
capturing a first cell type on the first set of electrodes on the first side of the membrane in the membrane exchange area.

13. The method of claim 12, wherein the first cell type is captured by dielectrophoresis.

14. The method of claim 12, wherein the first cell type comprises cardiomyocytes.

15. The method of claim 14, wherein the first cell type comprises induced pluripotent stem cell-derived cardiomyocytes (iPSC-CMs).

16. The method of claim 14, wherein the first cell type is electronically measured by the first set of electrodes.

17. The method of claim 12, wherein a second cell type is captured on the second set of electrodes on the second side of the membrane in the membrane exchange area.

18. The method of claim 17, wherein the second cell type comprises epithelial or endothelial cells.

19. The method of claim 17, wherein the second cell type comprises human umbilical vein endothelial cells.

20. The method of claim 17, wherein the second cell type is electronically measured by the second set of electrodes.
